(19) United States
(12) Patent Application Publication
(10) Pub. No.: US 2004/0047930 A1
(43) Pub. Date: Mar. 11, 2004

(54) Fortified nutmeg oil pain relief formulations

(75) Inventors: Joel H. Webbe, St. Georges (GD); Valentine Duncan, St. Georges (GD); Vaughan Forsyth, St. Davids (GD); Leonard St. Bernard, St. Georges (GD)

Correspondence Address: VENABLE, BAETJER, HOWARD AND CIVILETTI, LLP, P.O. BOX 34385, WASHINGTON, DC, 20043-9998, US

(73) Assignee: W & W Spices Grenada Ltd., St. Georges (GD)

(21) Appl. No.: 10/655,212

(22) Filed: Sep. 5, 2003

(51) Int. Cl. A61K 35/78

(52) U.S. Cl. 424/769

(57) ABSTRACT

A topical formulation and pharmaceutical compositions comprise nutmeg oil containing an intrinsic menthol component, and added menthol in an amount of at least about 0.1% by weight, the total menthol being in the range of 0.2% to 16% of the formulation by weight.

### BACKGROUND OF THE INVENTION

The present invention relates generally to topical pain relief formulations based on nutmeg extracts. More particularly the invention relates to a formulation comprising a nutmeg oil base fortified with natural menthol in an amount sufficient to provide penetrating pain relief.

Active ingredients frequently used in the topical or external pain relief formulations include camphor, capsaicin, menthol, and methyl salicylate.

Camphor is a white crystalline substance that may be obtained from the tree Cinnamonum camphora. However, nowadays more than three-fourths of the camphor sold in the United States is produced synthetically (usually from pinene). It is a local irritant, numbs the peripheral sensory nerves, and is slightly antiseptic. Camphor has a strong, penetrating, fragrant odor and a bitter, pungent taste. Frequently, its strong odor renders the camphor-containing pain relief products unpleasant and unacceptable to many users and persons nearby.

The drawback of capsaicin lies with its mechanism of action. Capsaicin relieves pain by depleting Substance P which delivers the pain message from the source of pain to the nervous system. Thus, it requires regular and consistent use of products such as ZOSTRIX® to be effective, i.e., 2-4 times a day for long periods of time in order to maintain a reduced level of Substance P. Similarly, the pain relief effect of capsaicin does not work right away and there can be a long delay in pain relief, a week or two for arthritic pain, two to four weeks for neuralgia pain, and up to six weeks for head and neck neuralgia pain. In addition, discomforts associated with capsaicin products include a burning sensation which is intolerable for some patients and leads to discontinuation of use.

Methyl salicylate, which occurs in wintergreen and sweet birch oil, is a counterirritant and can be made synthetically. The recommended topical dosage is a 10 to 60% concentration applied 3-4 times daily. Methyl salicylate readily penetrates intact skin after application. Because of the possibility of percutaneous absorption, methyl salicylate should be used with caution in individuals who are sensitive to aspirin or suffer from asthma or nasal polyps. Use of heating pads in conjunction with methyl salicylate or application after strenuous exercise, especially during hot and humid weather should be avoided. These conditions can magnify the extent of systemic absorption, and cause severe adverse reactions.

Menthol is a counterirritant. It is a secondary alcohol extracted from peppermint and can be made synthetically. In concentrations from 1.25 to 16 percent, menthol possesses counterirritant properties, producing a feeling of coolness followed by a feeling of warmth. Menthol induces vasodilation, causing the sensation of cold instead of actually cooling of the skin, and the skin temperature at the site is actually warmer than other parts of the body.

Several commercially available topical pain relief products contain one or more of the aforementioned ingredients.

For example, Ease Vaporizing Rub contains camphor 5.3%, menthol 2.8%, turpentine oil 4.8%, eucalyptus oil 1.5%, peppermint oil 1.5% and nutmeg oil 0.5%. The base for this formulation is not disclosed.

VICKS® VapoRub is a similar petroleum-based product, containing camphor 4.8%, menthol 2.6% and eucalyptus oil 1.2% as active ingredients. The inactive ingredients include cedar leaf oil, 0.5% nutmeg oil, spirits of turpentine, thymol, and special petrolatum.

Oriyen Green Balm contains undisclosed low percentages of nutmeg oil, menthol, peppermint oil, eucalyptus oil and methyl salicylate. The base of this product is not disclosed.

BEN-GAY®, ICY-HOT®, and Flexall contain methyl salicylate and menthol as the active ingredients. BEN-GAY® and Flexall also contain camphor as an active ingredient.

BLUE STUFF® and SUPER BLUESTUFF® are gel-based products containing aloe vera concentrate, water, menthol, glucosamine HCl, capsicum oleoresin, coriander oil, and various other ingredients.

Regular STOPAIN® spray contains just menthol as the active ingredient.

Easy Breathers is an impregnated tissue product, containing undisclosed percentages of camphor, menthol, methyl salicylate, nutmeg oil, and pine oil. DURA-PATCH®, THERAPATCH®, and ABSORBINE JR.® Pain Relief Patch contain either capsaicin, menthol, or both as the active ingredients.

NUT-MED is an alcohol-based spray not available in the United States, containing 14% nutmeg oil, 2% mace oil, 8% methyl salicylate, and 6% menthol. The alcohol and coconut oil base constitute about 70% of the composition. This product uses water-distilled nutmeg oil which is oxidized, hydrolyzed and not complete in its constituents.

There are quite a few other pain relieving formulations. However, not all of these products are effective, and they have other drawbacks.

### SUMMARY OF THE INVENTION

A novel fortified nutmeg oil formulation overcomes the unpleasant odors, discomfort, and limited efficacy associated with many of the aforementioned topical analgesic products. The fortified nutmeg oil formulation at a low concentration of menthol provides effective, penetrating pain relief comparable or superior to that of commercial topical analgesic products with higher concentrations of menthol and other analgesics. The fortified nutmeg oil formulation of the present invention also overcomes quality and effectiveness problems associated with many herbal-containing analgesic products.

The fortified nutmeg oil formulation can be prepared from natural botanical ingredients, and can be 100% organic. The nutmeg oil can be 100% pure. A preferred carrier (Grenada Nutmeg Oil) has penetrating characteristics that allow the fortified nutmeg oil formulation to be very fast acting. The purity of the nutmeg oil makes it gentle on the skin compared with other essential oils and carriers which can irritate the skin. It maximizes the effect of menthol, hence minimal quantities can be used to get similar or greater effects than other products with larger concentrations.

According to the invention, a topical formulation comprises at least about 25% of nutmeg oil containing an intrinsic menthol component, and added menthol in an amount of at least about 0.1% by weight, the total menthol being in the range of 0.2% to 16% of the formulation by weight.

The nutmeg oil may be prepared from Myristica fragrans and may be prepared by steam distillation. The nutmeg oil may be non-irritating nutmeg oil, containing less than about 5% of myristicin and less than about 0.5% of safrole. The nutmeg oil may be complete nutmeg oil, comprising intrinsic menthol, β-citronellol, bornyl acetate, and 1-t-carophyllene.

The formulation may comprise about 50% to about 99% nutmeg oil by weight, and about 0.2% to about 16% total menthol by weight. The formulation may comprise about 84% to about 99% nutmeg oil by weight, and about 0.2% to about 16% total menthol by weight. The formulation may consist essentially of nutmeg oil and added menthol, and the added menthol may be about 2% by weight.

The total menthol may be about 0.2% to about 1.0%, about 2%, about 1.25% to about 5%, about 5% to about 10%, about 10% to about 16%.

The formulation of claim 1, wherein the formulation consists essentially of nutmeg oil and added menthol

The formulation may be effective to relieve pain when applied topically to a subject.

The formulation may be contained in a container holding the formulation, with labeling associated with the container indicating use for treatment of pain. The labeling may indicate use for one or more of temporary relief of minor aches and pains of muscles and joints associated with simple backache, arthritis, bruises, sprains and strains; temporary relief of pain associated with rheumatism; temporary relief of aches and pains of muscles and joints associated with over-exercising & sports injuries; temporary relief of pain associated with bursitis, body aches and cramps; topical pain relief; topical fast acting pain relief, topical targeted pain relief; massage therapy; aches and pain associated with seniors; adjunct treatment of physical therapy; prescription drug and over the counter medication.

The formulation may comprise about 0.2% to about 1.0% total menthol, and the formulation is effective for the temporary relief of pain and/or itching associated with minor burns, sunburn, minor cuts, scrapes, insect bites, and/or minor skin irritations.

The formulation may comprise a non-nutmeg essential oil in an amount of less than about 2%. The non-nutmeg essential oil may comprise pimento berry (allspice) oil. The nutmeg oil and added menthol may be effective to relieve pain without additional non-menthol analgesics.

A method for treating pain comprises administering to a patient having pain, topically, a pain relieving formulation comprising nutmeg oil and added menthol, in an amount effective to relieve pain, without administering other topical analgesics.

A method for producing a topical formulation comprises selecting a target menthol level for the formulation in the range of 0.1% to 16% w/w, obtaining nutmeg oil, determining the amount of menthol equivalent in the intrinsic menthol component in the nutmeg extract, and if the amount of menthol equivalent is less than the target menthol level, adding extrinsic menthol to make up the difference, between the amount of menthol equivalent and the target menthol level, the total amount of menthol being in the range of 0.1% to 16%.

A pharmaceutical preparation comprises a container comprising at least about 25% nutmeg oil, the oil being non-irritating, and the preparation containing a predetermined concentration of menthol in the range of 0.1% to 16%, associated with labeling indicating use for relief of pain.

Further objectives and advantages, as well as the structure and function of preferred embodiments will become apparent from a consideration of the description, drawings, and examples.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

“Nutmeg oil” is an essential oil obtained from nutmeg using methods such as compression, solvent extraction, water distillation, steam distillation, and supercritical carbon dioxide extraction . “Complete nutmeg oil” means a nutmeg oil containing characteristic indicator constituents, such as citronellol, caryophyllene, and bornyl acetate. In determining completeness of nutmeg oil, gas chromatography may be used to detect the presence of these indicator compounds, rather than percentage.

“Non-irritating nutmeg oil” means nutmeg oil that contains low amounts of safrole and myristicin, other irritating constituents, oxidized constituents and byproducts, sufficient to satisfy regulatory or consumer acceptance requirements. For example, it contains less than about 5%, less than about 2%, less than about 1%, or less than about 0.6% or less than about 0.5% of myristicin, and less than about 3%, less than about 1%, preferably less than about 0.5% or less than about 0.3% safrole. Safrole and myristicin are suspected to be toxic and/or carcinogenic. Grenada nutmeg oil is preferred.

“Non-nutmeg essential oil” means an essential oil obtained from a plant other than nutmeg, such as eucalyptus or pimento berry oil.

“Menthol” as a chemical compound is intended to cover 5-methyl-2-(1-methylethyl)-cyclohexanol in an levorotatory (l), dextrorotatory (d) or racemic (dl) form. The menthol compound can be derived from natural sources or synthetically made. The l form is listed as Item No. 5882 in the Merck Index 12^{th} edition, Merck & Co., Inc, Whitehouse Station, N.J., 1996. Menthol USP as identified in the United States Pharmacopeia (USP) may be levorotatory, from natural or synthetic sources, or racemic.

“Menthol component” is used interchangeably with the term “intrinsic menthol component.” It is intended to cover menthol as a chemical compound, and the isomers, analogues such as cis-p-menth-2-en-ol, and salts or esters of menthol as a chemical compound that occur naturally in a nutmeg oil.

“Menthol equivalent” is intended to cover constituents in the intrinsic menthol component that are recognized as menthol as a chemical compound or an equivalent of menthol as a chemical compound by a person of ordinary skill in the art and/or the regulators.

“Extrinsic menthol” is used interchangeably with the term “added menthol.” It is intended to include menthol as a chemical compound, Menthol USP, and includes menthol extracts derived from plants of the mint family. A preferred example is the menthol extract of peppermint oil.

“Total menthol” is intended to cover the sum total menthol in the fortified nutmeg oil formulation, that is, the combination of added menthol and menthol equivalent in the menthol component of the nutmeg oil present in the formulation. For example, a nutmeg oil having 0.4% (w/w) of menthol equivalent together with 0.85% (w/w) of added menthol will result in a total menthol of 1.25% (w/w). For a formulation comprising 50% nutmeg oil having 0.4% menthol equivalent, the balance being another vehicle, the added menthol would need to be 1.05% to make up the same total menthol.

An “analgesic” or “anesthetic” is a topically (externally) applied drug that relieves pain by depressing cutaneous sensory receptors or as a topical counterirritant that stimulates cutaneous sensory receptors. Other mechanisms of action known or hereafter apparent to persons of ordinary skill are also intended to be included.

“Non-menthol analgesics” is intended to include methyl salicylate, camphor, bensocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphorated metacresol, juniper tar, phenol, phenolate sodium, resorcinol, diphenhydramine hydrochloride, tripelennamine hydrochloride, hydrocortisone, hydrocortisone acetate, allyl isothiocyanate, strong ammonia solution, turpentine oil, methyl nicotinate, capsaicin, capsicum, capsicum oleoresin, eugenol, pimento berry (Allspice) oil and other compounds recognized as safe and effective.

“Labeling associated with a container” includes writing printed on the container label, or a box around it, in printed materials sold with the container or displayed with the container, and/or marketing materials used to promote the material, on television, radio, the internet, or in print.

One of the advantages of using natural botanical ingredients for therapeutic purposes is that they frequently contain multiple constituents working synergistically to bring about the therapeutic effects. Individually each constituent may exist in a very small percentage, but the combined contribution of the constituents as a whole toward the therapeutic effects of the natural botanical ingredient can be much more significant. However, more often than not, the quality and property of the natural ingredients and their interactions can affect their therapeutic effects. Influential factors include, but are not limited to, plant species, cultivation location and condition, harvest method and timing, handling and processing method, and storage condition. Thus, controlling and maintaining a consistent high quality of natural botanical ingredient is of utmost importance for providing a safe and effective medication. This is not an easy task because of the many variables involved, especially when some of the factors, e.g., weather, are uncontrollable. It requires tremendous efforts not only to optimize all the aforementioned factors to obtain a high quality natural botanical ingredient, but also to continue to produce, control and maintain the natural ingredient at a consistent, high quality.

The present invention takes advantage of natural botanical ingredients by providing a novel pain relief formulation comprising a quality-controlled natural botanical ingredient, nutmeg oil.

The use of nutmeg can be dated back for centuries. It has been used as food flavor and spice and as treatment for some gastrointestinal discomforts. However, pure nutmeg oil has been considered a toxic substance even when it is applied topically. It has generally been diluted in a carrier before application or used in very small quantity. This is evident in the aforementioned nutmeg oil-containing products in which nutmeg oil constitutes to only a minor portion of the products, less than 1%. The toxic constituents in nutmeg oil have been identified to be myristicin and safrole.

One source for nutmeg is Myristica fragrans Houtt of the Myristicaceae family. Common names and synonyms of nutmeg include mace, muscadier, muskatbaum, myristica, nuez moscada, nux moschata, and such species as Myristica officinalis L., Myristica argentea (Macassar Mace), Myristica malabarica (Bombay Mace), and Myristica moschata. Nutmeg is the kernel of the apricot-like fruit of the nutmeg tree and enclosed in a hard seed-case covered with an arillus. The soft membranous coat is mace. Nutmeg oil is a volatile oil also known as oleum myristicae, ol. Myrist, myristica oil, essence de muscade, atheririsches muskatol, essencia de moscada and essencia de nuez moscada. It is similar to the volatile oil of mace and sometimes no commercial distinction is made between the two.

The present invention enables the use of nutmeg oil in high concentration, by using nutmeg oil with low concentrations of safrole and myristicin.

About 30-55% of the nutmeg seed consists of oils and 45-60% consists of solid matter including cellulose materials. Nutmeg oil, or “essential oil of nutmeg,” also called the “volatile oil” accounts for 5-15% of the nutmeg seed and nutmeg butter, the “fixed oil of nutmeg,” sometimes called expressed oil of nutmeg, accounts for 24-40% of the nutmeg seed, depending on geographical origin.

The essential oil obtained by steam distillation is a colorless or pale yellow liquid with a taste and odor of nutmeg. This oil is soluble in alcohol and insoluble in water and may have a density of 0.859-0.924 g/mL at 25° C. Since it is light and air sensitive, it should be kept cool in a tightly closed container and protected from light.

According to “Nutmeg and derivatives” FO: MISC/94/7 (FAO, Rome, 1994) the major components of an example of the essential oil and their relative percentages are as listed below in Table 1. There is disagreement between the Merck Index and others as to which of sabinene or camphene comprises about 50% of the essential oil:

TABLE 1
Quantities of nutmeg oil components

| Component | Relative Percentage |
|---|---|
| sabinene or camphor | (50%) |
| d-pinene | 20% |
| dipentene | 8% |
| d-linalool | 6% |
| d-borneol | 6% |
| i-terpineol | 6% |
| geraniol | 6% |
| myristicin | 4% |
| safrole | 0.6% |
| eugenol | 2% |
| iso eugenol | 2% |

A qualitative listing of components that were observed in the essential oil along with their classification based on type is given below in Table 2:

TABLE 2
Nutmeg oil components by class

| Class | Components |
|---|---|
| Aromatic ethers | methyl eugenol, eugenol, methyl iso-eugenol, |
| | myristicin, methoxy eugenol, elemicin, safrole, |
| | iso-eugenol, iso-elemicin |
| Terpenes | α-terpinene, myrcene, γ-terpinene, terpinolene, |
| | α-pinene, camphene, β-pinene, uinonene |
| | (dipentene), α-phellandrene, sabinene, β- |
| | phellandrene, δ-carene, α-thujene |
| Monoterpene Alcohol | geraniol, 4-terpineol, α-terpineol, β-terpineol, |
| | citronellol, linalool |
| Sesquiterpene | Caryophyllene |
| Terpinic Esters | geranyl acetate, linalyl acetate, bornyl acetate |
| Acids | formic acid, octanoic acid, butyric acid, acetic |
| | acid |
| Aromatic hydrocarbons | p-cymene, toluene |

The following compounds were identified on the basis of retention times observed from gas chromatography: cumene, cyclamen aldehyde, camphor, menthone and menthyl isovalerate.

It can be observed that citronellol, caryophyllene, and bornyl acetate are useful indicator compounds for several of the more useful classes of compounds.

As illustrated in Table 3, nutmeg oil samples obtained from nutmeg trees grown in different countries contained different levels of constituents. Among the constituents, cis-p-Menth-2-en-ol is an analog of the menthol compound. These constituents and their analogs, including eugenol, methyl eugenol, methylisoeugenol, methoxy-eugenol, isoeugenol, camphor, menthone, cumene cyclamen aldehyde, menthyl isovalerate, borneol, geraniol, 4-terpineol, alpha-terpineol, citronellal and linalool may be found and may contribute at various degrees toward the analgesic effects of nutmeg oil. Eugenol is a well-known anesthetic and widely used in dentistry, due to its analgesic, antiseptic and balsamic qualities. Eugenol can also combine with methylisoeugenol, methoxy-eugenol and isoeugenol to give a synergistic effect. Myristicin and safrole contribute to the toxic effects. The organoleptic properties of nutmeg oil are directly related to the origin of the raw material. East Indian oils (Indonesian oils) are higher in myristicin, up to 13.5%, compared with less than 1% in West Indian oils (Grenada), which are low in alpha pinene, safrole and myristicin, but higher in sabinene. For nutmeg oil from Indonesia, the benefits from having higher contents of eugenol, methyl-eugenol, and cis-p-menth-2-en-ol are offset by its much higher contents of myristicin and safrole. Accordingly, a preferred nutmeg oil base is the Grenada nutmeg oil.

TABLE 3
Constituents of nutmeg trees grown in different countries

| | | | | | | | Sin- |
|---|---|---|---|---|---|---|---|
| | Gre- | St. | Malay | Pap- | Indo- | Pe- | ga- |
| Component | nada | Vincent | Seedlings | ua | nesia | nang | pore |
| Alpha-Pinene | 10.6 | 12.6 | 12.8 | 21.3 | 18.0 | 19.9 | 21.2 |
| Camphene | 0.2 | 0.2 | 0.2 | 0.3 | 0.3 | 0.3 | 0.4 |
| Beta-Pinene | 7.8 | 12.1 | 9.3 | 14.3 | 9.7 | 17.7 | 12.7 |
| Sabinene | 50.7 | 49.6 | 44.1 | 30.0 | 27.0 | 36.3 | 17.8 |
| Myrcene | 2.5 | 2.8 | 2.9 | 2.4 | 2.2 | 2.5 | 2.6 |
| Alpha- | 0.4 | 0.6 | 0.6 | 0.5 | 0.5 | 0.4 | 1.0 |
| Phellandrene | | | | | | | |
| Alpha- | 0.8 | 1.9 | 1.8 | 1.1 | 2.0 | 0.8 | 4.0 |
| Terpinene | | | | | | | |
| Limonene | 3.1 | 3.3 | 3.1 | 2.7 | 2.7 | 2.8 | 3.6 |
| 1,8-Cineole | 2.5 | 2.3 | 2.1 | 1.9 | 1.8 | 1.5 | 3.2 |
| Gamma- | 1.9 | 3.1 | 2.8 | 1.9 | 3.3 | 1.3 | 6.8 |
| Terpinene | | | | | | | |
| P-Cymene | 3.2 | 0.7 | 0.8 | 0.5 | 0.7 | 0.3 | 1.8 |
| Terpinolene | 1.7 | 1.2 | 1.2 | 1.1 | 1.1 | 0.6 | 2.1 |
| Trans- | 0.8 | 0.3 | 0.5 | 0.1 | 0.6 | 0.6 | 0.3 |
| Sabinene- | | | | | | | |
| hydrate | | | | | | | |
| Copaene | 0.3 | * | * | 0.2 | 0.3 | * | 0.2 |
| Linalol | 0.9 | 0.4 | 0.2 | 1.0 | 0.3 | 0.2 | 0.8 |
| Cis-Sabinene- | 0.7 | 0.2 | 0.4 | 0.2 | 0.6 | 0.2 | 0.2 |
| hydrate | | | | | | | |
| Cis-p-Menth- | 0.4 | 0.1 | 0.1 | 0.3 | 0.5 | 0.1 | 0.3 |
| 2-en-ol | | | | | | | |
| Terpinen-4-ol | 6.1 | 3.5 | 6.0 | 3.9 | 7.3 | 2.0 | 9.3 |
| Cis-Piperitol | 0.5 | 0.4 | 0.4 | 0.6 | 0.4 | 0.3 | 0.5 |
| Safrole | 0.2 | 0.1 | 0.8 | 1.5 | 2.1 | 0.6 | 1.9 |
| Methyl-eugenol | 0.2 | 0.1 | 0.5 | 0.2 | 1.2 | 0.6 | 0.6 |
| Eugenol | 0.2 | * | 0.3 | 0.1 | 0.7 | 0.3 | * |
| Elimicin | 1.4 | 1.3 | 1.7 | 0.4 | 0.5 | 4.6 | 0.3 |
| Myristicin | 0.5 | 0.8 | 4.1 | 10.4 | 13.5 | 3.3 | 6.3 |

Data obtained from Sookram, R. “The effect of drying temperature on the quality and quantity of nutmeg essential oil” The nutmeg research program, 1990. Dept. of Chemical Engineering UWI St. Augustine Campus Trinidad.

Table 4 illustrates the physical properties of two types of nutmeg oil, mainly East Indian Nutmeg Oil and West Indian Nutmeg Oil. The East Indian Nutmeg Oil has a density of 0.885 to 0.915 g/mL and is soluble in 90% alcohol at a ratio of 1 part oil to 3 parts alcohol. West Indian Nutmeg Oil has a density of 0.86-0.88 g/mL and is soluble in 90% alcohol at a ratio of 1 part oil to 4 parts alcohol. (information obtained from www.inchem.org/documents/pims/plant/pim355.htm).

TABLE 4
Comparison of the physical properties of East Indian nutmeg oil
(Indonesian oil) and West Indian nutmeg oil (Grenada oil)

| Characteristic | Grenada oil | Indonesian oil |
|---|---|---|
| Specific gravity (25° C.) | 0.854-0.880 | 0.880-0.930 |
| Refractive index (20° C.) | 1.469-1.480 | 1.474-1.488 |
| Optical rotation | +20 to +45° | +2 to +30° |
| Solubility (v/v % alcohol) | 1:4 (90%) | 1:3 (90%) |

By selecting the best species of the nutmeg trees, careful cultivation in optimal environment, proper handling and collection methods, optimal extraction technique, monitoring of the key constituents, and optimal storage condition, a high quality, stable, non-irritating nutmeg oil can be obtained and used in pharmaceutical formulation in high percentage. A preferred nutmeg oil is GNO from W& W Spices.

Unexpected advantages result from using fortified high quality nutmeg oil, that is, using nutmeg oil as a carrier for menthol. The menthol equivalents contained within the intrinsic menthol component of the nutmeg oil can reduce the amount of added menthol needed to achieve a specific amount of total menthol in the fortified formulation. Other active constituents in the nutmeg oil of the present invention also work synergistically with the added menthol to bring about a penetrating pain relief. Unexpectedly, a fortified nutmeg oil formulation of the present invention containing a low percentage of total menthol was found to be as effective as or superior to commercial topical pain relief products containing higher percentages of menthol compound in addition to methyl salicylate and camphor in providing pain relief and with superior aroma, appearance, and consistency as it is rubbed into the skin. Thus, the fortified nutmeg oil formulation of the present invention provides a superior alternative for pain relief.

The present invention relates to fortified nutmeg oil formulations as over-the-counter external analgesic drug products in a form suitable for topical administration. The formulations may be used to treat pain associated with many conditions by topically applying the compositions to the area of pain. The formulations of the present invention provide penetrating pain relief. The formulations of the present invention can be used for temporary relief of minor aches and pains of muscles and joints associated with simple backache, arthritis, bruises, sprains and strains; temporary relief of pain associated with rheumatism; temporary relief of aches and pains of muscles and joints associated with over-exercising & sports injuries; temporary relief of pain associated with bursitis, body aches and cramps; topical pain relief; topical fast acting pain relief, topical targeted pain relief; and massage therapy; as a prescription drug or over-the-counter drug, or as a botanical. The formulations may be used in dentistry, particularly with added eugenol or eugenol-containing materials such as pimento berry oil.

One embodiment of fortified nutmeg oil formulations of the present invention contains 0.1-1% of menthol and has a topical analgesic effect, possibly by depressing cutaneous sensory receptors. Another embodiment of fortified nutmeg oil formulations contains 1.25-16% of menthol and produces a cooling sensation, and may have a topical counterirritant effect by stimulating cutaneous sensory receptors.

The fortified nutmeg oil formulations of the present invention can be labeled as an external analgesic, topical analgesic or pain relieving oil formulation. The 0.1%-1.0% menthol formulation can also be labeled for the temporary relief of pain, itching, or both, associated with minor burns, sunburn, minor cuts, scrapes, insect bites, or minor skin irritations.

The formulations of the present invention may be used to treat pain caused by sports related injuries. Such sports-related injuries include, but are not limited to, hematomas, bruises, sprains (e.g., ankle sprain), muscle spasms (e.g., pulled muscles), partial tendon tears, tendonitis, bursitis, myositis, traumatic arthritis and post-insertion of joint dislocation. In treating pain associated with sports related injuries, the formulations of the present invention are applied to the area of pain as described herein. The formulations may be used in combination with sports-injury therapy techniques such as physical therapy, acupuncture, weight-training, biofeedback techniques, among others.

The present formulations may also be used in treating pain unique to senior citizens. Much of the bone,joint or muscle pain experienced by seniors results from a combination of sources. Some of these sources are known, others are not. In certain cases, such pain is a natural consequence of the diseases resulting from the aging process, which includes pain accompanied with diminished motor function, atrophy, dietary changes, among others. Using the present formulations to treat bone, joint or muscle pain in seniors can be effective in minimizing the amount of pain relief medication they already take, or would be required to take in the future. Also, pain in seniors contributes to depression, inactivity and immobility in this age group. Diminution in pain resulting from use of the fortified nutmeg oil formulations would result in greater independence, increased activity, socialization, appetite and overall sense of well-being in an elderly patient.

In addition, the formulations of the present invention can be utilized as an adjunct to physical therapy. Generally, physical therapy involves passive and active treatments or methodologies to strengthen and/or heal muscles, tendons, bones, and joints. The draw backs of physical therapy include pain and discomfort to the patient. The formulations of the present invention can be used to treat such pain. For example, the present formulation may be applied to the area of pain before, during, and/or after each physical therapy treatment.

The preparation method of the present invention starts from the species selection of nutmeg trees. The preferred species is Myristica fragrans Houtt. The nutmeg trees are cultivated at locations most suitable for their growth. Mature nutmeg can be harvested by picking from the trees or collecting those falling from the trees. After cleaning and inspection, the nutmegs can be dried to a certain moisture content, for example, about 10%. Nutmeg oil is then collected from the dried ground nutmegs, by a suitable method, for example by steam distillation or supercritical carbon dioxide extraction.

Preferably, the complete nutmeg oil is maintained in a controlled environment to ensure that (1) the oil remains complete, that is, no components are missing as monitored via gas chromatography; (2) the oil is not oxidized; (3) the oil has good characteristic nutmeg aroma; (4) the clarity of the oil is maintained; (5) irritants are at sufficient low concentrations; and (6) the menthol component is as desired.

Specifically, a topical pain relief formulation according to the invention using only naturally occurring organic compounds, non-irritating nutmeg oil and the addition of appropriate amount of menthol. Nutmeg oil can be referred to as the carrier or vehicle. Its penetrating ability allows menthol (a proven topical analgesic) to reach the source of pain surprisingly quickly.

The total menthol composition can range from 1.25% as stated above to a maximum of 16.00% (w/w) with the corresponding nutmeg oil content ranging from 98.9% to 84.00% (w/w) respectively. Other embodiments have 0.1 to 1.0% total menthol, or 0.1% to 16% (w/w) of total menthol. The nutmeg oil can be more than about 25%, more than about 50%, more than about 75%, or more than about 84% (w/w) of the formulation with the difference in weight made up by essential oils and other oils from vegetables, fruits, nuts, etc. Prior topical formulations used much lower concentrations of nutmeg oil, typically below 1%. Contrary to conventional experience, use of the higher concentrations is desirable. Using less than about 25% nutmeg oil however results in some loss of the advantages of the invention.

### EXAMPLES

### Example 1

### Preparation of Nutmeg Oil

W & W Spices GNO Grenada nutmeg oil was obtained. Nutmeg was harvested from Grenada nutmeg trees after the fruit matured and was dried to a moisture content of approximately 10% (w/w). Nutmeg oil was obtained by steam distillation with purification.

The nutmeg oil passed the following standards:

| | |
|---|---|
| Refractive index (20° C.): | 1.472-1.477 |
| Specific density (20° C.): | 0.840-0.910 |
| Myristicin: | <5.0% |
| Safrole: | <0.5% |

Organoleptic Analysis:

color—colorless to pale yellow

odor—fresh, light and sweet spicy odor characteristic of

nutmeg

clarity—clear and clean without traces of water or suspended matter

### Example 2

### Gas Chromatography (GC) Analysis of Nutmeg Oil

A batch of pure nutmeg oil prepared according to Example 1 was analyzed using GC. The contents of some key constituents were also quantified. Briefly, the analytical test was carried out on Agilent 6890 which was equipped with a capillary column under the test conditions: Oven temperature at 50° C., detector temperature at 250° C., and measuring time of 32 minutes. The mobile phase was nitrogen. A sample of 5% menthol USP was prepared and used as a reference standard.

Batches of pure nutmeg oil contained the following key constituents: β-citronellol (at 15.38 min), bomyl acetate (at 16.91 min), and 1-t-caryophyllene (at 20.55 min). They also contained 0.1% to 0.2% menthol, 0.1%-0.3% safrole and 0.4% to 0.6% myristicin.

### Example 3

### Preparation of Fortified Nutmeg Oil Formulation

Grenada Nutmeg Oil (GNO) from Example 1 was combined with USP grade menthol prepared from natural peppermint. The menthol crystals were combined with the nutmeg oil in an agitated mixing vessel. The mixture was agitated until the menthol crystals are fully dissolved and the mixture was homogenous. A GC analysis was then performed to verify that the weight percentage of menthol was correct as expected. The end product, W & W Spices' GNO, was composed of an average of 1.25% (w/w) menthol, including the average 0.15% menthol naturally present in the nutmeg oil and 1.1% added menthol (natural plant extracted USP grade menthol), in 98.9% (w/w) steam distilled nutmeg oil from Grenada.

Another formulation had 5.0% total menthol, more menthol being added.

Another formulation had 2.0% total menthol.

### Example 4

### Pain Relief Effect Compared With a Currently Marketed Topical Analgesic Product

A fortified nutmeg oil formulation containing 1.25%(w/w) topical menthol in Grenada nutmeg oil was topically applied to voluntary subjects and an equal amount of BEN-GAY® Ultra strength was also topically applied to the same voluntary subjects.

Surprisingly, the pain relief of 1.25%(w/w) total menthol in Granada nutmeg oil was as effective in relieving pain as BEN-GAY® Ultra strength, containing 30% methyl salicylate, 10% of menthol and 4% of camphor. In addition, the fortified nutmeg oil formation took effect faster than BEN-GAY® Ultra strength. Subjects also preferred fortified nutmeg oil formulation for its aroma and pleasant texture/consistency.

This suggested that not only nutmeg oil was a superior carrier for menthol, but also worked synergistically with the added menthol to provide penetrating pain relief at a low level of total menthol.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A topical formulation comprising:
at least about 25% of nutmeg oil containing an intrinsic menthol component, and
added menthol in an amount of at least about 0.1% by weight,
the total menthol being in the range of 0.2% to 16% of the formulation by weight.

2. The formulation of claim 1, wherein the nutmeg oil is prepared from Myristica fragrans.

3. The formulation of claim 1, wherein the nutmeg oil is prepared by steam distillation.

4. The formulation of claim 1, wherein the nutmeg oil is non-irritating nutmeg oil, containing less than about 5% of myristicin and less than about 0.5% of safrole.

5. The formulation of claim 1, wherein the nutmeg oil is complete nutmeg oil, comprising intrinsic menthol, β-citronellol, bornyl acetate, and 1-t-carophyllene.

6. The formulation of claim 1, wherein the formulation comprises about 50% to about 99% nutmeg oil by weight, and about 0.2% to about 16% total menthol by weight.

7. The formulation of claim 1, wherein the formulation comprises about 84% to about 99% nutmeg oil by weight, and about 0.2% to about 16% total menthol by weight.

8. The formulation of claim 1, wherein the formulation consists essentially of nutmeg oil and added menthol.

9. The formulation of claim 1, wherein the total menthol is about 0.2% to about 1.0%

10. The formulation of claim 1, wherein the total menthol is about 2%.

11. The formulation of claim 1, wherein the total menthol is about 1.25% to about 5%.

12. The formulation of claim 1, wherein the total menthol is about 5% to about 10%.

13. The formulation of claim 1, wherein the total menthol is about 10% to about 16%.

14. The formulation of claim 1, wherein the formulation consists essentially of nutmeg oil and added menthol, the added menthol being about 2% by weight.

15. The formulation of claim 1, being effective to relieve pain when applied topically to a subject.

16. The formulation of claim 1, further comprising a container holding the formulation, and labeling associated with the container indicating use for treatment of pain.

17. The formulation of claim 16, wherein the labeling indicates use for one or more of temporary relief of minor aches and pains of muscles and joints associated with simple backache, arthritis, bruises, sprains and strains; temporary relief of pain associated with rheumatism; temporary relief of aches and pains of muscles and joints associated with over-exercising & sports injuries; temporary relief of pain associated with bursitis, body aches and cramps; topical pain relief; topical fast acting pain relief, topical targeted pain relief; massage therapy; aches and pain associated with seniors; adjunct treatment of physical therapy; prescription drug and over the counter medication.

18. The formulation of claim 1, wherein the formulation comprises about 0.2% to about 1.0% total menthol, and the formulation is effective for the temporary relief of pain and/or itching associated with minor burns, sunburn, minor cuts, scrapes, insect bites, and/or minor skin irritations.

19. The formulation of claim 1, wherein the formulation further comprises a non-nutmeg essential oil in an amount of less than about 2%.

20. The formulation of claim 19, wherein the non-nutmeg essential oil comprises pimento berry (allspice) oil.

21. The formulation of claim 1, wherein the nutmeg oil and added menthol are effective to relieve pain without additional non-menthol analgesics.

22. A method for treating pain comprising administering to a patient, topically, a pain relieving formulation comprising nutmeg oil and added menthol in an amount effective to relieve pain, without administering other topical analgesics.

23. A method for producing a topical formulation comprising:
selecting a target menthol level for the formulation in the range of 0.1% to 16% w/w,
obtaining nutmeg oil,
determining the amount of menthol equivalent in the intrinsic menthol component in the nutmeg extract, and
if the amount of menthol equivalent is less than the target menthol level, adding extrinsic menthol to make up the difference between the amount of menthol equivalent and the target menthol level,
the total amount of menthol being in the range of 0.1% to 16%.

24. A pharmaceutical preparation comprising a container comprising at least about 25% nutmeg oil, the oil being non-irritating, and the preparation containing a predetermined concentration of menthol in the range of 0.1% to 16%, associated with labeling indicating use for relief of pain.
